(19) United States
(12) Patent Application Publication
(10) Pub. No.: US 2024/0293022 A1
(43) Pub. Date: Sep. 5, 2024

(54) SYSTEM AND METHOD FOR STEREOSCOPIC VISION TESTING

(71) Applicant: Eyes in Sync Inc., Oakland, CA (US)

(72) Inventors: Maureen Kennedy Powers, Oakland, CA (US); Neil Kaapuni, Playa Del Rey, CA (US); Yoshitomo Moriwaki, Westchester, CA (US)

(21) Appl. No.: 18/595,250

(22) Filed: Mar. 4, 2024

Related U.S. Application Data
(60) Provisional application No. 63/449,615, filed on Mar. 3, 2023.

(51) Int. Cl. A61B 3/08; A61B 3/00; A61B 90/50; A63F 13/52; H04N 13/15; H04N 13/344

(52) U.S. Cl. CPC A61B 3/085; A61B 3/005; A61B 3/0091; A63F 13/52; H04N 13/15; H04N 13/344; A61B 2090/502; A63F 2300/8082

(57) ABSTRACT

A vision testing system that allows a user to execute a set of virtual reality (VR) vision testing applications with stereoscopic and shimmering effect is disclosed. The system includes a communication device, a VR gaming unit, a wireless input remote controller and a stereoscopic VR viewing device. The controller enables the user to provide VR input signals to a VR gaming application installed in the communication device. The VR viewing device splits a display screen of the communication device into a left image portion and a right image portion and directs the image data displayed at the left image portion and at the right image portion to the left eye and to the right eye of the user respectively. The stereoscopic VR viewing device to view a set of VR games with stereoscopic and shimmering effect in perception induced by a binocular rivalry feature of the eyes of the user.

### PRIORITY CLAIM

This application claims priority from the U.S. provisional application with Ser. No. 63/449,615, which was filed on Mar. 3, 2023, the disclosure of which is incorporated herein as if set out in full.

### BACKGROUND OF THE DISCLOSURE

### Technical Field of the Disclosure

The present invention relates generally to vision testing platforms, and more particularly, to a virtual reality (VR) system utilizing binocular rivalry that enables the user to play a set of VR games that utilizes stereoscopic and shimmering effects across two eyes as a means for vision testing.

### Description of the Related Art

Virtual Reality (VR) systems have become well known and common in the last ten years, and often present truly immersive first-person perspectives for the users of the system. The users experience and influence the game environment through a variety of VR devices and accessories, including VR headsets, sensor-equipped gloves, hand controllers and adjustable 3D VR glasses. Virtual reality is generally a self-controlled environment, where the user can control the simulated environment via a system. Virtual reality can also enhance a fictional environment through the use of sensors, displays and other features like motion tracking, movement tracking etc. The users immerse themselves in a specifically designed simulated environment for a specific purpose such as medical training and gaming etc.

A stereoscopic image making is a technique which is used to enable a three dimensional effect, thereby adding to an illusion of depth to a flat image. It is the visual perception of differential distances between or among objects in one's line of sight. A stereoscopic viewfinder in one embodiment partially obstructs the user's vision so that each eye only sees one image. The system can then display a subtly separate image to each eye, allowing the user to “perceive” depth just one would in the real world. Various VR systems and programs are available and most of them provide a basic kit that includes hardware components such as a VR headset with input controllers and an adjustable 3D VR glasses.

There is a need for a virtual reality system which would be designed to capitalize on the effect stereoscopic imagery and moving objects may have on a user. Such a system would provide a controllable and adjustable wireless input remote device and would include specific program or applications tailored for the specific testing of various abilities of the human eyes, brain, and brain-eye connection. The system would utilize the binocular rivalry feature which would explore the shimmering effect in perception when different colors or stimuli are presented to corresponding areas of the retina in both eyes. Moreover, such a virtual reality system would be equipped to manipulate the user's brain and resulting perceptual effect could be detected externally via input commands input to the program in response to the user's observations within the system. Similarly, such a system would allow the user to play a set of virtual reality vision testing games utilizing a stereoscopic display device designed to provide a three-dimensional effect in a computer generated virtual environment.

### SUMMARY OF THE INVENTION

To minimize the limitations found in the prior art, and to minimize other limitations that will be apparent upon the reading of the specification, the present disclosure provides a vision testing system that allows a user to play a set of Virtual Reality (VR) game with stereoscopic and shimmering effect in perception induced by a binocular rivalry feature of the eyes of the user. The vision testing system includes a communication device, a wireless input remote controller and a stereoscopic VR viewing device. The communication device includes at least one display screen, but in some embodiments may comprise at least two separate display screens. In the preferred embodiment, the communication device is a smartphone running Android mobile operating system and the single display is split into two images.

In summary, the invention relates to a vision testing system designed to leverage virtual reality (VR) technology to provide an immersive environment for vision testing. This system comprises a communication device equipped with at least one display screen, which is operably connected to a VR unit. The VR unit is enhanced by an application server residing on a central computer, which is equipped with a processor and a memory unit. The memory unit is integrated with a central database, facilitating a robust framework for the VR vision testing application, which includes a suite of VR vision testing games or applications.

The VR unit is a pivotal component of the system, hosting a VR vision testing application that resides on the central computer. This application server is a sophisticated platform that manages the deployment of various VR vision testing scenarios, facilitating a dynamic and interactive visual testing environment. The processor installed on the central computer orchestrates the operation of the VR applications, ensuring seamless performance and real-time responsiveness, while the associated memory unit stores the necessary data, including user responses and test results, in the central database.

The VR unit is operably connected to the communication device via a network. The VR unit includes a server residing on a central computer having a processor on which is installed with a VR testing application and coupled with a memory unit integrated with a central database. The VR gaming application may preferably include multiple testing applications.

The wireless input remote controller is physically or wirelessly coupled to the communication device. The input remote controller enables the user to provide VR input signals to the VR application installed in the communication device and modifies image data associated with the set of VR testing games displayed at the display screen based on the VR input signals provided. The input remote controller is portable such that the user can hold the input remote controller in their hand while they are viewing/playing the games installed in the communication device.

The stereoscopic VR viewing device is configured to connect with the communication/computing device utilizing a securing member. The stereoscopic VR viewing device enables the user to perform stereoscopic vision testing on the device. Preferably, the device may be a single screen mobile phone, but in some instances the stereoscopic VR viewing device includes one or more screens directly. In this embodiment no mobile phone or other communication device is required.

The VR vision testing application comprises a set of specialized applications or games, each designed to test different aspects of visual function, such as depth perception, color vision, peripheral awareness, and reaction to visual stimuli. These applications are integrated into the VR environment, providing a comprehensive platform for vision testing that is both engaging and clinically informative. The VR viewing device preferably splits the mobile phone display screen into a left image portion and a right image portion and directs the image data displayed at the left image portion and at the right image portion to the left eye and to the right eye of the user respectively. The stereoscopic VR viewing device partially obstructs the user's vision such that each eye only sees one image. Preferably, the stereoscopic VR viewing device may be any generic VR adapter such as Google Cardboard or Homido VR viewer. Google Cardboard and the Homido VR device simply split a middle portion of a mobile phone display screen into left and right images, which are then specifically directed to the left and right eye of the viewer. To be clear, the stereoscopic VR viewing device is capable of delivering a left image to a left eye and right image to a right eye of the user, regardless of whether it is a simple split screen or two separate screens.

This salient feature of the stereoscopic VR viewing device enables the user to view the set of VR testing games with a stereoscopic and shimmering effect in perception induced by a binocular rivalry feature of the eyes of the user. The shimmering effect in perception occurs when two different colors are presented to corresponding areas of the retina in each of the two eyes. In the preferred vision testing system, the shimmering and the stereoscopic effects are beneficially utilized.

The system further comprises a VR game including a grid sword game and clock sword game. The system further comprises a VR headset with an integrated display screen adapted to present separate images to each eye. In embodiments, the VR game is a module within a VR gaming application, the VR gaming application configured to execute the at least one VR game and track the user's progress and responses within the central database. Further, the least one VR game comprises a coordination game that requires the user to align objects within a virtual space, the coordination game configured to enhance the user's hand-eye coordination and spatial awareness within the VR environment. In other embodiments, the at least one VR game comprises a reaction time challenge that presents at least a first visual stimulus in the user's peripheral vision, in addition to puzzles that promote depth perception by requiring the arrangement of objects at varying virtual distances. In further embodiments, the reaction time challenge VR game assesses and trains the user's peripheral vision response and depth discrimination abilities. In some embodiments, the display screen comprises at least two separate display screens, and/or wherein the display screen comprises an LCD screen, and/or wherein the display screen comprises an LED screen.

In some embodiments, a stereoscopic vision testing system is disclosed comprising: a display mechanism capable of presenting a first visual stimulus to a user's left eye, and at least a second visual stimulus to the viewer's right eye; the first visual stimulus and at least second visual stimulus providing the user the ability to detect distinct images in a virtual reality (VR) environment; wherein the system leverages the user's ability to merge the first visual stimulus and the at least second visual stimulus into at one unified picture; and wherein the VR environment comprises a virtual reality (VR) unit operably connected to the communication device, the VR unit including an application server residing on a central computer having a processor on which is installed with a VR vision testing application and coupled with a memory unit integrated with a central database, the VR vision testing application comprising a set of VR vision testing applications.

Said stereoscopic vision testing system includes detection by the user, the detection being determined by the user's response in a psychophysical paradigm wherein eye movements are not measured. Said psychophysical paradigm may include a choice reaction time test. The VR system may include a VR headset with an integrated display screen for presenting the separate images to each eye. A first visual stimulus and at least second visual stimulus of the system may comprise dynamic images, said dynamic images presenting a user movement within the VR environment. In other embodiments, the first visual stimulus and at least second visual stimulus comprise two rivalrous images. In some embodiments, an input device is included that is adapted to record the user's responses.

Further disclosed is a method for generating a shimmering effect for multimedia applications, comprising the steps of: presenting a first visual stimulus to a user's left eye; simultaneously presenting at least a second visual stimulus to the viewer's right eye; and wherein the first visual stimulus and the at least second visual stimulus are configured such that the user's brain cannot resolve the first visual stimulus and second visual stimulus into a single coherent image, thereby producing binocular rivalry and the perception of shimmer in the user. In embodiments, the first visual stimulus may include a first color and the at least second visual stimulus comprises at least a second color, wherein the perception of shimmer is utilized in video or film special effects to enhance visual content. In some embodiments, the first color comprises a primary color and/or wherein the first visual stimulus and the at least second visual stimulus comprise complementary colors to enhance the perception of shimmer. In other embodiments, the first visual stimulus and the at least second visual stimulus include geometric shapes and/or wherein the first visual stimulus and the at least second visual stimulus comprise a narrative in video or film. Further embodiments include a control mechanism to adjust the intensity of stimulus presented.

A first objective of the present embodiment is to provide a vision testing system with a stereoscopic virtual reality viewing device that enables a user to execute a number of virtual reality vision testing games with a stereoscopic and shimmering effect in perception induced by a binocular rivalry feature of the eyes of the user.

A second objective of the present embodiment is to provide a virtual reality system that allows the user to play vision testing games utilizing a stereoscopic display device designed to provide a three-dimensional effect in a computer-generated virtual environment.

A third objective of the present embodiment is to provide a vision testing system featuring a stereoscopic VR viewing device that splits a display screen into a left image portion and a right image portion and directs the image data displayed at the left image and at the right image portion to the left eye and to the right eye of the user respectively.

These and other advantages and features of the present invention are described with specificity so as to make the present invention understandable to one of ordinary skill in the art.

### BRIEF DESCRIPTION OF THE DRAWINGS

In order to enhance clarity and improve understanding of these various elements and embodiments of the invention, elements in the figures have not necessarily been drawn to scale. Furthermore, elements that are known to be common and well understood to those in the industry are not depicted in order to provide a clear view of the various embodiments of the invention. Thus, the drawings are generalized in form in the interest of clarity and conciseness.

FIG. 1 illustrates normal stereoscopic vision, where the eyes naturally converge and diverge, depending on the distance of the target, to attain a single image in the brain;

FIG. 2 illustrates a manipulation that can result in binocular fusion of separate images in the two eyes, to form a uniform single image in the brain. illustrates how VR can be used to test for stereo vision;

FIG. 3 illustrates a block diagram of a vision testing system in accordance with the preferred embodiment of the present invention;

FIG. 4 illustrates a front perspective view of an exemplary communication device connected to a stereoscopic VR viewing device connected to a communication device for the vision testing system in accordance with the preferred embodiment of the present invention;

FIGS. 5A-5C illustrate examples of stereopsis. FIG. 5A illustrates an example of stereopsis in accordance with a preferred embodiment of the present invention. FIG. 5B illustrates an adjustable example of stereopsis in accordance with a preferred embodiment of the present invention. FIG. 5C illustrates the user screen during vergence adjustment.

FIGS. 6A-6C illustrate examples of different vergence adjustment tasks in accordance with the preferred embodiment of the present invention.

FIG. 7 illustrates an example of binocular rivalry between the eyes occurs when different colors monocular stimuli are presented to each eye and as the brain attempts to resolve this binocular rivalry effect;

FIGS. 8A-8B illustrate different chromatic input to the different eyes to prompt a binocular rivalry;

FIGS. 9A-9C illustrate further embodiments of combining left and right images to each eye;

FIGS. 10A-10C illustrate an example of a spatial three-dimensional vision testing in accordance with the preferred embodiment of the present invention;

FIGS. 11A-11C illustrate an example of a game element utilized in the vision testing system in accordance with the preferred embodiment of the present invention.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized, and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term ‘about” means +/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

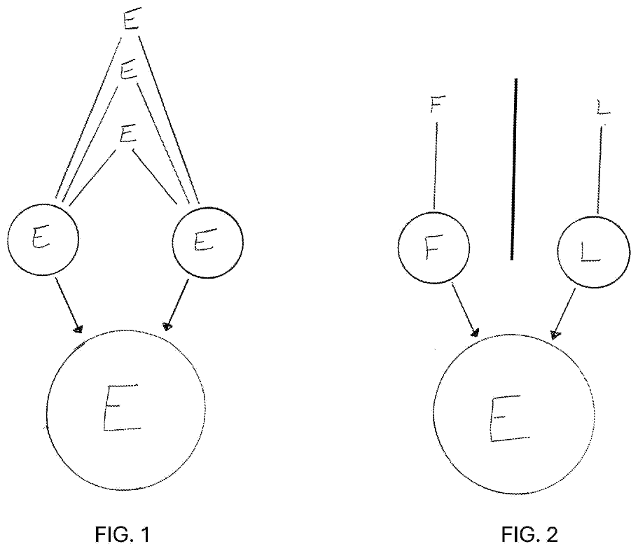

In embodiments, FIG. 1 illustrates normal 3D vision (also referred to herein as “stereoscopic vision”), wherein a vision target appears in both eyes and those images are fused in the brain to produce a sensation of depth. The viewer in this case has no VR headset or any guidance as to how to move the eyes so that both converge on the same target. Humans must learn this skill, which normally starts to develop in about the 5^{th} month of life. To the extent that one is not able to control vergence, the image in the brain is not uniform and cannot accurately indicate depth. Illustrated is the hypothetical perception of a single visual target, the capital letter E, in free space. If the viewer moves their eyes to converge accurately as the E moves closer and farther away, then stereo vision occurs, and the image is seen in depth. If not, then there may appear to be 2 E's, located in different regions of space.

In embodiments, FIG. 2 shows that VR separates the images in the eyes from each other (dotted line). If the letter E were again presented to each eye, a viewer with good stereo would again perceive a single E because they are able to bring the images into convergence through eye movement control. In the illustration, two different images are presented to each eye: a letter F to the left eye and a letter L to the right. If the viewer can manipulate their eyes to bring the images together—and only if they can do so—they will see an E, which is the fused image of the F and L. If they perceive anything other than an E, poor stereo vision is indicated.

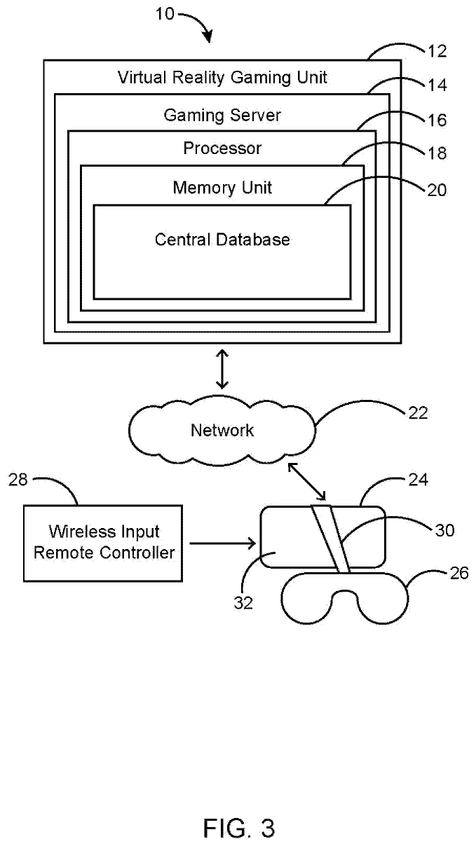

Referring to FIGS. 3 and 4, a vision testing and entertainment system 10 that allows a user to play a set of virtual reality (VR) vision testing games 12 with stereoscopic and shimmering effects is illustrated. The vision testing system 10 preferably includes a communication device 24, a virtual reality (VR) unit 12, a wireless input remote controller 28 and a stereoscopic VR viewing device 26. The communication device 24 includes at least one display screen 32. In the preferred embodiment, the communication device 24 is a smartphone running the Android mobile operating system, but the system may use iOS operating system or any suitable operating system capable of displaying one screen or two screens. The VR unit 12 may be operably connected to the communication device 24 via a network 22. The VR unit 12 includes an application server 14 residing on a central computer having a processor 16 on which is installed a VR gaming application and coupled with a memory unit 18 integrated with a central database 20. The VR gaming application preferably includes a set of VR vision testing games.

Interaction with the VR environment is enabled through a wireless input remote controller 28, which is coupled to the communication device 24. The input remote controller 28 enables the user to provide VR input signals to the VR gaming application installed in the communication device 24 and modifies image data associated to the set of VR vision testing programs displayed at the display screen 32 based on the VR input signals provided. The modifications in image data, prompted by user inputs, are reflected in the VR games displayed on the screen, enabling a tailored testing experience that can adapt to various user actions and preferences. In the preferred embodiment, the user may hold the input remote controller 28 in their hand while they view/play the vision testing games installed in the communication device 24. Preferably, the input remote controller 28 is available in two configurations: a mobile VR and a headset VR. In the mobile VR, a smartphone serves as a VR headset. Here, the user requires some sort of external controller. In an instance of the input controller for the mobile VR, the system 10 is compatible with every Bluetooth controller for smartphones. And in the case of headset VR, the headsets are integrated with the input controllers 28. Certain prominent brands of headset VR are HTC VIVE®, Oculus Rift® and Meta Quest Pro®. Preferably, each VR platform implements its own game controller 28.

In embodiments, the VR headset may include an OLED or LCD display that providing visuals essential for an immersive virtual reality experience. In embodiments, the VR headset field of view may span around 100 to 110 degrees, mimicking the natural human range of vision, enhancing the user's sense of presence within a virtual environment. In some examples, high refresh rates of 90 Hz or more ensure a smooth and responsive experience, reducing motion blur and minimizing the risk of motion sickness. In some examples, advanced inside-out tracking systems employ cameras and sensors built into the headset, eliminating the need for external sensors and allowing for a seamless setup. Integrated spatial audio enriches the immersive experience with 3D sound, while ergonomic design and adjustable straps ensure comfort during extended use.

The system includes a stereoscopic VR viewing device, designed to provide a binocular view wherein each eye is presented with a distinct image or a set of images. This device is crucial for inducing a stereoscopic effect, which is essential for the depth perception tests embedded within the VR games. As shown in FIGS. 3 and 4, the stereoscopic VR viewing device 26 is configured to connect to the communication device 24 utilizing a securing member 30. The stereoscopic VR viewing device 26 enables the user to perform stereoscopic vision testing on the communication device 24. The VR viewing device 26 preferably splits the display screen 32 into a left image portion and a right image portion and directs the image data displayed at the left image portion and at the right image portion to the left eye and to the right eye of the user respectively. In this way, the stereoscopic VR viewing device 26 partially obstructs the user's vision such that each eye only sees one image. In other embodiments, two separate screens are used and may be integrated directly into a headset (not shown).

As discussed above, the VR viewing device 26 as shown in FIG. 4 or sometimes referred generally herein as a “VR system” includes a VR headset with an integrated display screen for presenting the separate images to each eye. In some embodiments, the VR headset is equipped with an integrated display screen specifically designed to present separate images to each eye, creating a stereoscopic effect. The integrated display screen is optimized to deliver high-resolution images that facilitate the testing of 3D vision by providing clear, distinct images to the left and right eyes separately. In some embodiments, the headset is designed to comfortably fit different head sizes and shapes while ensuring that the display screens are correctly aligned with the user's eyes. In further embodiments, the VR headset includes adjustable straps and cushioning to enhance comfort during prolonged use. The stereoscopic effect is achieved through specialized software that controls the display of images, ensuring synchronization between the user's movements and the visual presentation for an immersive experience. This setup is crucial for accurately assessing the user's ability to perceive depth and detect a uniform image from two separate images presented.

The display screens may be embodied in various forms to cater to different aspects of the virtual reality experience and vision testing procedures. These variations include, but are not limited to, liquid crystal displays (LCDs) which provide a sharp and persistent visual output, light-emitting diode (LED) screens known for their brightness and energy efficiency, organic LED (OLED) screens which offer deep black levels and high contrast ratios, active-matrix OLED (AMOLED) displays that support faster refresh rates and are beneficial for quick visual response testing, flexible OLED screens allowing for curved display surfaces to enhance field-of-view, quantum dot LED (QLED) screens that deliver a wider color spectrum, e-ink displays which are effective for low-power usage scenarios and provide a paper-like reading experience, microLED displays that offer high resolution and pixel density for detailed visual testing, digital light processing (DLP) projectors for large-scale vision testing environments, and holographic displays which can create three-dimensional images without the need for specialized glasses, providing a unique depth perception challenge in vision testing. These display screen embodiments can be selectively implemented within the communication device of the vision testing system to optimize the visual stimuli presented to the user based on the specific requirements of the vision test or therapeutic application.

This unique feature of the stereoscopic VR viewing device 26 enables the user to view the set of VR vision testing programs with a stereoscopic and shimmering effect in perception induced by a binocular rivalry feature of the eyes of the user. Preferably, the stereoscopic VR viewing device 26 may be any generic VR adapter such as Google Cardboard or Homido VR viewer. Google Cardboard and the Homido VR device simply split a middle portion of a mobile phone display screen 32 into left and right images, which are then specifically directed to the left and right eyes of the viewer respectively. To be clear, the stereoscopic VR viewing device 26 can deliver a left image to a left eye and a right image to a right eye of the user. In one embodiment, the securing member 30 is an opaque barrier preventing one eye from seeing the view intended for the other eye. The stereoscopic nature of the VR vision testing application and the stereoscopic VR viewing device 26 allows the user to play the set of VR games 12 with stereoscopic and shimmering effect. The shimmering effect in perception occurs when two different colors are presented to corresponding areas of the retina in each of the two eyes. In the preferred vision testing system 10, the shimmering and the stereoscopic effects are beneficially utilized.

In embodiments of the disclosed vision testing system, VR games include a multitude of interactive applications and scenarios designed to evaluate and enhance various aspects of stereoscopic vision. These may include but are not limited to basic coordination games that require the user to align objects within a virtual space, reaction time challenges where users must respond to stimuli that appear in the periphery of their vision, puzzles that encourage depth perception by having users arrange objects at different virtual distances, adventure games that guide users through a narrative while presenting them with depth-based tasks, educational applications that train users in recognizing subtle shifts in depth and color, sports simulations that replicate the depth cues of real-world activities, therapeutic exercises aimed at improving ocular motor skills, dynamic tracking games that adjust to the user's eye movements to test and train focus agility, memory games that use stereoscopic cues to enhance cognitive links to visual stimuli, and multiplayer games that allow for collaborative or competitive vision testing and training in a socially interactive virtual environment. In embodiments, said VR games enhance user engagement while simultaneously assessing and exercising the visual faculties of the user, contributing to a comprehensive vision testing system.

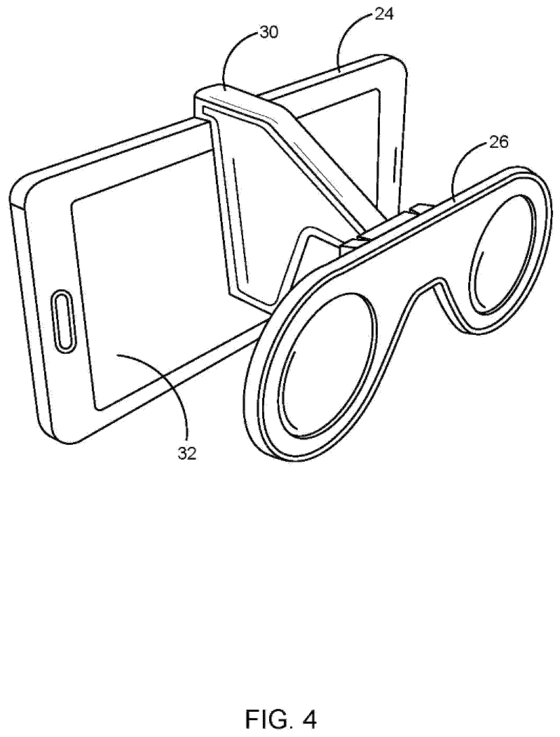

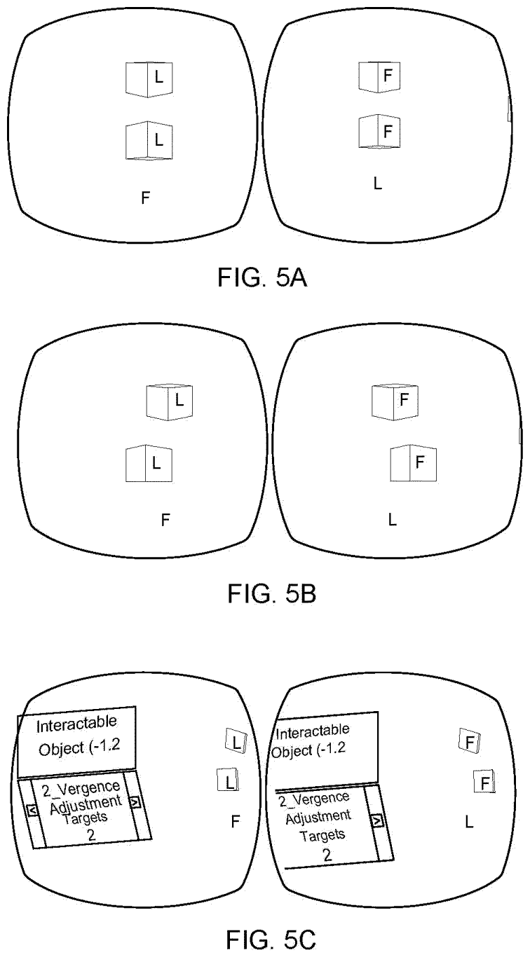

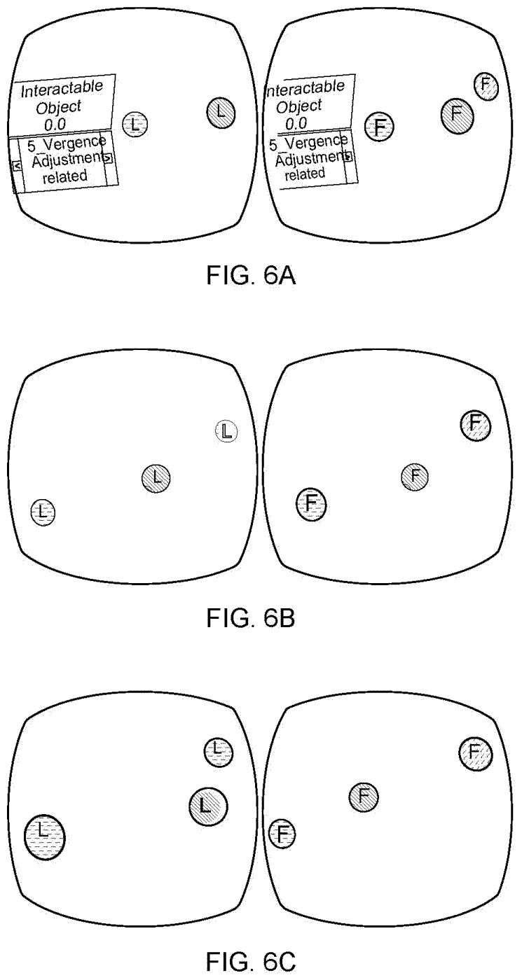

As depicted in the testing system screenshots shown in FIGS. 5-11, in certain embodiments a method and system for testing 3D (stereoscopic) vision is disclosed, based on a person's ability to detect a uniform image when two separate images (or two rivalrous images) are presented to each in VR eye display. Detection is determined by the user's response in a psychophysical paradigm. In some embodiments, eye movements are not measured. In other embodiments, the VR system may present static images to each eye separately. These images are carefully designed to test the user's stereoscopic vision without the need for dynamic content, which may introduce variables such as motion parallax. The static images may include a range of patterns, symbols, or simple objects that require the user to focus and discern depth to detect a uniform image. In certain embodiments, this allows for a controlled testing environment where variables can be systematically altered to assess different aspects of stereoscopic vision. The static nature of the images also serves to reduce the potential for VR-induced motion sickness, making the testing process more comfortable for users who may be sensitive to moving visuals.

As depicted in the screenshots of FIGS. 5-11, in other embodiments the separate images presented to each eye are dynamic, including elements of movement within the VR environment to create a more challenging test of stereoscopic vision. The dynamic images can simulate real-life scenarios where objects move across the user's field of vision at various speeds and trajectories, thus requiring the user to adjust their focus and vergence in real-time. In some embodiments, this dynamic presentation is intended to mimic the complexities of visual perception in natural settings, providing a robust assessment of the user's ability to maintain a uniform image amid changing visual stimuli. The movement patterns of the objects can be programmed to test various levels of stereoscopic vision, from basic to advanced, and can be adjusted according to the user's performance.

In certain embodiments, such as that shown in FIGS. 5A-5C stereopsis is the subject. FIG. 5A illustrates an example of stereopsis in accordance with a preferred embodiment of the present invention. FIG. 5B illustrates an adjustable example of stereopsis in accordance with a preferred embodiment of the present invention. FIG. 5C illustrates the user screen during vergence adjustment. In embodiments, the user screen and accompanying system may include an input device adapted to record the user's responses. Said input device to record the user's responses are operably employed during the stereoscopic vision testing. In embodiments, this input device may take the form of a handheld controller, voice recognition system, or a touch-sensitive interface integrated into the VR headset. The device captures the user's input signals as they respond to the visual tests, providing immediate feedback to the system. The recorded responses are then analyzed to determine the accuracy and speed of the user's ability to detect a uniform image from the two separate images presented. In embodiments, the input device is ergonomically designed to be easily used by the user without breaking immersion, ensuring a seamless testing experience.

FIG. 5 illustrates an example of stereopsis in accordance with a preferred embodiment of the present invention. Stereopsis is the ability to join two images from the two eyes into one perceived image in the brain as opposed to experiencing rivalry because the two images cannot be joined.

As shown in FIG. 5A, the user should see an L with the left eye and an F with the right eye, which combined creates the visual effect of an E in each of the 3 images as depicted in FIGS. 1 and 2. If not, there is an indication of weakness in eye coordination to achieve fusion, for the brain's understanding. As shown in FIG. 5B, the user must change the vergence angle (the degree to which the eyes move toward or away from the nose) in order to see the E-combined from an L in one eye and an F in the other—for each of the three images. In embodiments, FIG. 5C illustrates the user screen during vergence adjustment.

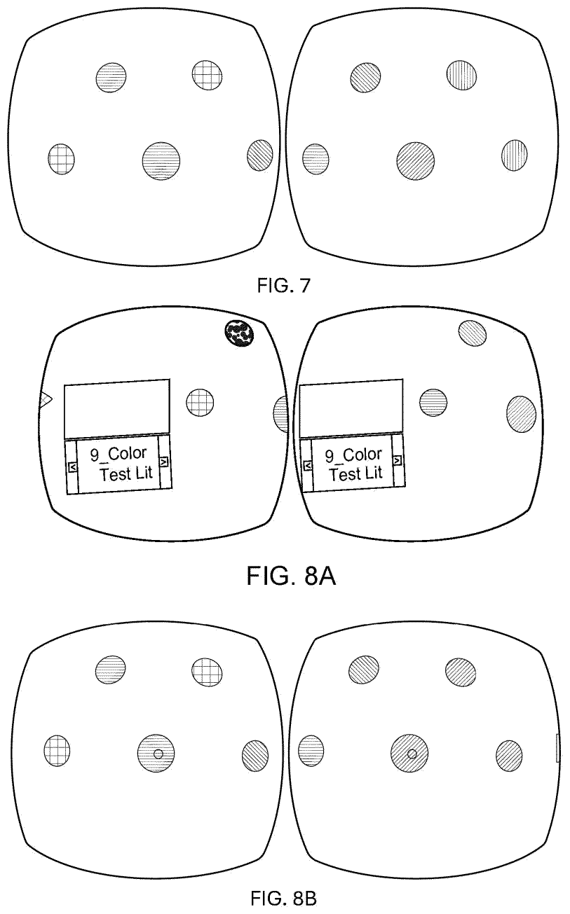

In embodiments, FIG. 7 illustrates an example of binocular rivalry between the eyes with different chromatic input in accordance with the preferred embodiment of the present invention.

As shown in FIG. 7, the binocular rivalry effect occurs when different colors (represented by different types of shading for purposes of this patent application) of monocular stimuli are presented to each eye and as the brain attempts to resolve this binocular rivalry effect. The user can experience a shimmering effect while the brain is trying to make sense of the two images—which is not a typical phenomenon. Usually, our eyes receive corresponding information about the world from the two eyes-just slightly offset, which gives the ability to perceive 3D (stereopsis). In this case, the images do not in all cases match, thus the brain can become confused as to what it is processing—but the perceptual effect is arresting and may have use in the fields of entertainment, gaming and importantly, testing. The degree of shimmering effect can be controlled by the color aspect of each stimulus. Typically, the farther away the colors are from each other on the color wheel, the larger the effect would be. Thus, the perceptual effect can easily be manipulated from mild to severe.

In one example, the psychophysical paradigm employed within the stereoscopic vision testing system incorporates a choice reaction time test to assess the user's stereoscopic vision capabilities. In embodiments, during this test users are presented with multiple visual stimuli and are required to quickly and accurately respond to the stimuli that match a given criterion, such as a specific shape or color that appears uniform when fused correctly by the user's stereoscopic vision. The system measures the time taken from the presentation of the stimuli to the user's response, providing data on the user's reaction time and accuracy. These metrics are then used to evaluate the user's depth perception and the ability to detect a uniform image when two separate images are presented to each eye. The choice reaction time test is customizable with various difficulty levels to challenge the user appropriately and track improvements over time.

In another example, the psychophysical paradigm employed within the stereoscopic vision testing system (also referred to herein as the “VR system”) incorporates a choice reaction time test to assess the user's stereoscopic vision capabilities. During this test, users are presented with multiple visual stimuli and are required to quickly and accurately respond to the stimuli that match a given criterion, such as a specific shape or color that appears uniform when fused correctly by the user's stereoscopic vision. The system measures the time taken from the presentation of the stimuli to the user's response, providing data on the user's reaction time and accuracy. These metrics are then used to evaluate the user's depth perception and the ability to detect a uniform image when two separate images are presented to each eye. The choice reaction time test is customizable with various difficulty levels to challenge the user appropriately and track improvements over time.

In some embodiments, FIGS. 8A-8B illustrates different chromatic input to the different eyes to prompt a binocular rivalry. In particular, an example of a test for stereo vision using binocular rivalry is shown. This method for generating a perceptual shimmering effect for multimedia applications comprising the steps of: a) presenting a first visual stimulus of to a user's left eye; and b) simultaneously presenting a second visual stimulus to the viewer's right eye; wherein the first visual stimulus and second visual stimulus are configured such that the user's brain cannot resolve the first visual stimulus and second visual stimulus into a single coherent image, thereby producing rivalry and the perception of shimmer in the user. Different color values (RGB values) are presented to each eye in VR. Depending on the RGB values, users may experience more or less rivalry. In some embodiments, the first visual stimulus comprises a first color and the second visual stimulus comprises a second color, and wherein the perception of shimmer is utilized in video or film special effects to enhance visual content. The test determines the presence or absence of rivalry or “shimmer”-if any is seen it indicates good stereo function. FIGS. 8A-8B illustrate examples of differing chromatic input to each eye prompting binocular rivalry. FIG. 8A illustrates an example of a perceptual effect due to the binocular rivalry of colors. Because of the perceptual effect, each region within the retinal placement in the two eyes is competing for brain space. The user can manipulate the color, amount, and placement of the retinal stimuli in VR to create whatever effect they desire.

FIG. 8B illustrates an example of the shimmering effect, using partial retinal stimuli to each eye. The dots in FIG. 8B represent the focus point for the user. FIG. 8B illustrates demonstration of shape independence in producing the shimmering effect plus keeping the user's eye on the central figure, which still appears to shimmer. Stereo vision can be tested by varying not only the RGB values of the focal circle, but also by varying the luminosity of each. In one embodiment, users are given the ability to adjust the RGB and/or luminosity values to achieve maximum shimmer.

In some embodiments, the first visual stimulus and the at least one second visual stimulus comprise primary colors, enhancing the vibrancy of the shimmering effect. In some embodiments, the stimuli are presented in complementary colors, which are known to enhance the shimmering effect due to their stark contrast when perceived together. In further embodiments, the choice of colors can be made dynamically to fit the thematic elements of the multimedia content in which the effect is being utilized. In further embodiments, the stimuli may encompass geometric shapes to add a structural element to the shimmering effect. In other examples, the geometric shapes may correspond to thematic or narrative elements within a video or film, thus integrating the visual effect into the story in a manner that is both aesthetic and functional. In yet another embodiment, the inclusion of shapes is tailored to the narrative context, enhancing the storytelling through visually engaging elements.

In another embodiment, the method includes a control mechanism that allows for the adjustment of the intensity of the colors presented in the stimuli. In some embodiments, this mechanism can be manual or automated, providing users or system operators with the ability to modulate the shimmering effect according to specific scenes or desired visual outcomes. In other embodiments, the control mechanism is responsive to the content being displayed, dynamically adjusting the stimuli to maintain the optimal shimmering effect throughout various segments of multimedia applications.

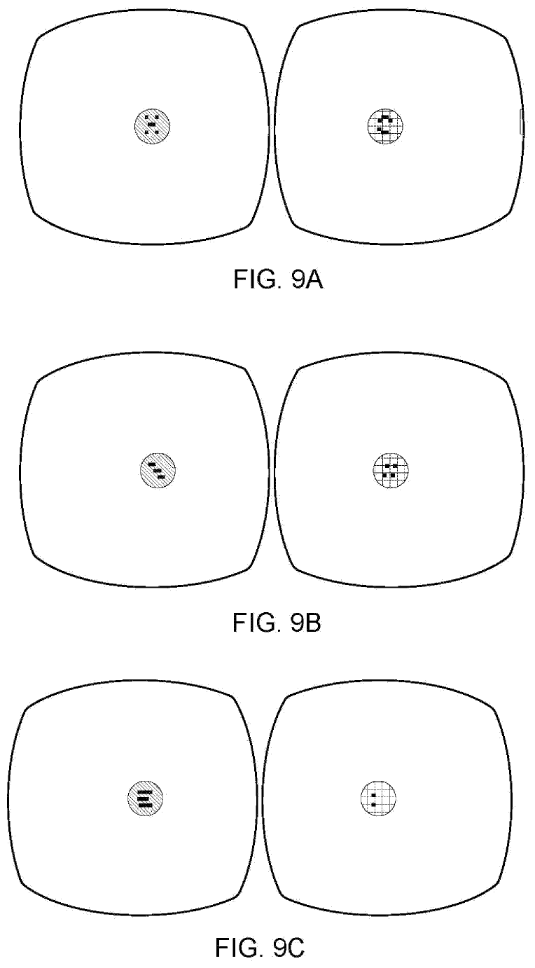

FIG. 9 shows a further example of combining different images in each eye, in this case to create for the perception of the user the letter “E”, but in this case using single pixels to formulate the “letter” in each eye, and wherein errors in convergence cause binocular rivalry.

FIGS. 9A and 9B Show examples of pixels that cannot be matched, and thus may produce rivalry as the attempt to attain binocular fusion fails. FIG. 9C Shows a pixelated E, with part of the image presented to the left eye and part to the right eye. When the eyes are properly aligned, the user will see a letter “E”. The task is more difficult because the pixels are so small; when the eyes are not aligned, the user perceives a broken-up E, with the right eye's dots floating near the left eyes, but not making a clear E.

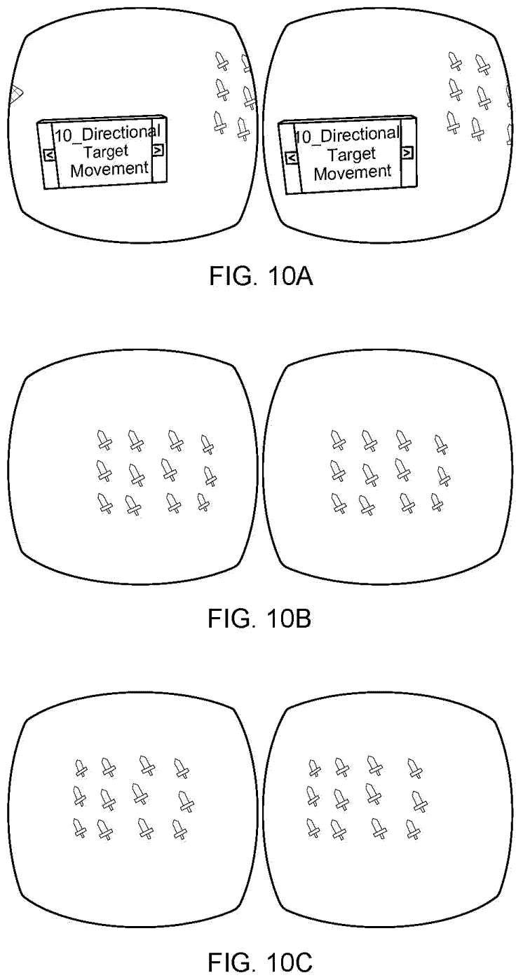

FIGS. 10A-10C illustrate an example of a spatial three-dimensional vision testing system in the form of the VR game termed “grid sword game” wherein target detection within a moving field is shown The task requires convergence and tracking skills. As shown in FIG. 10A, the grid sword game comprises several swords that move into spatial targets rather than color. In this grid sword game, the user must identify the sword that is different in depth from all the others. That sword can only be identified if it is viewed by both eyes together. In addition, this is a tracking test where all objects are moving in a circle centered in front of the player. The objects do not move independent of each other, and the purpose is to see how such movement affects the user's ability to identify the sword. A sign indicator shown in FIG. 10A reads “Directional Target Movement” and indicates that movement is being added, creating another level of difficulty for the user. In FIGS. 10B and 10C the field of swords is moving in a circular fashion, with the third sword from the left on the top moving at a different rate form the others. The user's task is to attain and maintain convergence, so all swords remain single images, as they are moving. Thus, the user is actually changing convergence slightly to continue to keep the grid stable.

In FIG. 10B, one of the swords appears spatially closer to the user. This is detectable by the user if the user's vergence angle is appropriate. The control of and measurement of eye position is completely based on perceptual/behavioral response. In the spatial three-dimensional vision testing, eye movement is not measured. The movement is constant and present in both eyes. For each, two frames are presented at different times. Here, there are two frames of a moving object taken one second apart. Because the 3D effect can only be appreciated if the user's eyes are properly positioned, we can use the fact that 3D is perceived by the user as evidence of eye position. As shown in FIG. 10C, all targets have moved after one second of moving in a circle.

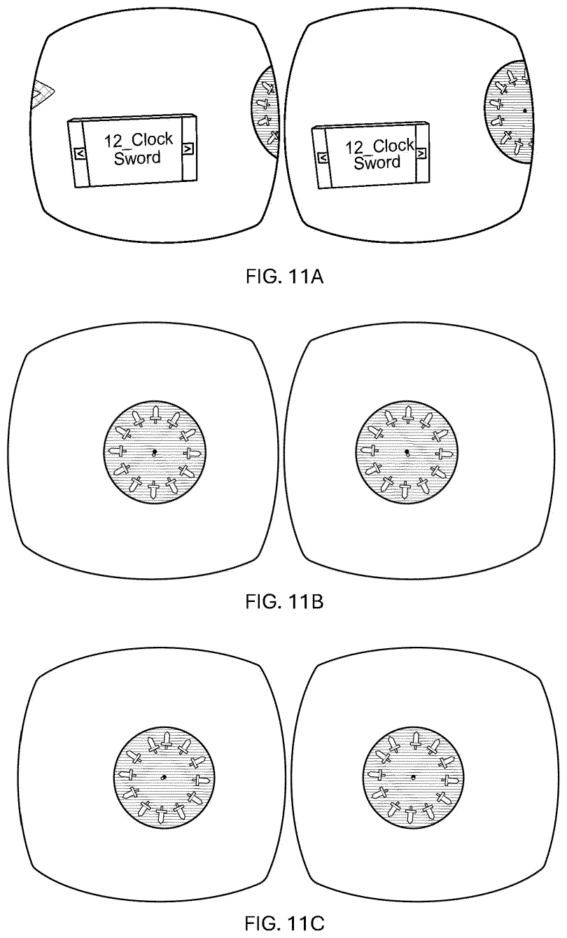

Relatedly, FIGS. 11A-11C illustrate an example of a VR game termed “clock sword game”. FIG. 11A indicates the name of the task. As shown, a sign indicator reads “12_Clocksword” and the circle (“clock face”) anchors a field of twelve swords. Only one is displaced, and as will be described, the user must have accurate convergence in order for it to be detected. In a “clock sword game”, the user must identify the sword that is different. That sword can only be identified if it is viewed by both eyes together. Utilizing this game element, the system is able to record and deliver metrics on the success rate to either the user or to the technician administering the testing. These same metrics regarding how many people were able to use the 3D cue may also be delivered over a network. As shown in FIG. 11B, no user could identify the spatially separated sword because at this stage all swords are at the same depth. Said again, none of the swords are displaced in this example. In FIG. 11B, the sword is identifiable. FIG. 11C shows an example of a unique sword depth, at 11 o'clock, resolvable only with the eyes converged appropriately. The sword will be seen in 3D by the user and will stand out in space if accurate convergence is attained. Otherwise, the image will appear double. For the purposes of this document, which is viewable only in two dimensions, the spatially separated sword is shown in FIG. 11C as slightly misaligned.

Further to the above, in embodiments FIGS. 1-11 show a method and system for testing 3D (stereoscopic) vision, based on a person's ability to detect a uniform image when two separate images (or two rivalrous images) are presented to each in VR. Detection is determined by the user's response in a psychophysical paradigm; eye movements are not measured. FIGS. 1-11 further show a method and system for producing a shimmering effect for use in video or film special effects, whereby different colors of stimuli are presented to each eye with the result that the brain cannot resolve the two colors into one, thus producing rivalry and a resulting perception of colorful shimmer (also referred to herein as “shimmer” or “shimmering”).

In some embodiments, the system includes a processor configured to adjust the separate images based on user feedback, allowing for real-time calibration of the stereoscopic effect. This processor can fine-tune the images to challenge the user's stereoscopic vision, making the system useful for both testing and training purposes. The system's software can record the adjustments and user responses for later analysis or for adapting future tests. In other embodiments, the system is integrated with an application that provides a variety of test patterns and environments, offering a comprehensive assessment of the user's stereoscopic vision. This application includes a library of images and scenarios, ranging from simple patterns to complex scenes, to test different aspects of 3D perception.

In still other embodiments, the system can provide a score or assessment report based on the user's performance in detecting uniform images, which could be used by optometrists, coaches, educators, or other professionals, or for personal monitoring of visual capabilities. The assessment includes metrics such as reaction time, accuracy, and consistency over multiple tests. In one example, the system includes an eye comfort feature that adjusts brightness and contrast levels to minimize eye strain during prolonged use. The system's software ensures that the images presented do not cause discomfort or fatigue, promoting a user-friendly experience while maintaining test effectiveness. In another example, the system is capable of wireless operation, enhancing the portability and ease of use. The VR headset communicates with the base system through a secure wireless connection, allowing users to move freely without being encumbered by cables.

In yet another embodiment, the system includes a tutorial mode that guides the user through the process of using the system and understanding the principles of stereoscopic vision. This mode is designed to educate users and ensure they are properly prepared for the testing procedure. In embodiments, the system is equipped with a calibration tool that customizes the testing protocol to the individual user's interocular distance and visual preferences. This tool ensures that the stereoscopic images are optimally aligned for each user, providing a personalized testing experience.

In some embodiments, the system provides real-time feedback to the user, offering tips and guidance to improve their ability to detect uniform images, which can be particularly beneficial for users undergoing vision therapy or rehabilitation. In other embodiments, the system is designed to integrate with other diagnostic tools, allowing for a comprehensive visual health assessment when used in conjunction with devices that measure other aspects of visual function. In still other embodiments, the system's software includes an algorithm that adapts the difficulty level of the test based on the user's performance, ensuring that the system remains challenging and effective for users with varying levels of stereoscopic vision proficiency.

As described above, in some embodiments the VR system comprises a VR headset with an integrated display screen. This VR headset is designed to present separate images to each eye of the user, thereby creating a stereoscopic effect that enhances the immersive experience of the VR environment. The integrated display screen within the headset can be of various technologies such as OLED, LCD, or other suitable display technologies that are capable of high refresh rates and resolution to provide a clear and responsive visual experience.

In certain embodiments, VR game(s) comprise a module within a VR gaming application, the VR gaming application configured to execute the at least one VR game and track the user's progress and responses within the central database. This application is engineered to not only execute various VR games but also to monitor and record the user's progress and responses. The central database integrated with the application stores this data, allowing for the tracking of improvements in vision and other cognitive abilities as the user interacts with different games within the application. The system's ability to track user progress is integral to its design. By recording responses and outcomes within the central database, the system enables longitudinal monitoring of visual function, allowing for the assessment of changes over time. This feature is particularly beneficial for tracking the progression of visual acuity, peripheral vision, and other eye health indicators in response to ongoing treatment or as part of routine eye health maintenance.

In other embodiments, the suite of VR games includes a coordination game module that necessitates the user to align objects within a virtual space. This game is specifically tailored to enhance the user's hand-eye coordination and spatial awareness, providing a training environment that adapts to the user's performance and becomes progressively more challenging to promote cognitive development within the VR environment. Another VR game is a reaction time challenge. This challenge presents visual stimuli within the user's peripheral vision and comprises puzzles that foster depth perception through the arrangement of objects at various distances in the virtual space. Such games are designed to engage different visual and cognitive skills, providing a varied and comprehensive visual training experience.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention to not be limited by this detailed description, but by the claims and the equivalents to the claims appended hereto.

What is claimed is:

1. A vision testing system comprising:
a communication device having at least one display screen;
a virtual reality (VR) unit operably connected to the communication device, the VR unit including an application server residing on a central computer having a processor on which is installed with a VR vision testing application and coupled with a memory unit integrated with a central database, the VR vision testing application comprising a set of VR vision testing applications;
a wireless input remote controller coupled to the communication device, the input remote controller enabling the user to provide VR input signals to the VR gaming application installed in the communication device and which modifies image data associated with the set of VR games displayed at the display screen based on the provided VR input signals; and
a stereoscopic VR viewing device configured to connect with the communication device, the VR viewing device having a left image portion and a right image portion and directs the image data displayed at the left image portion and at the right image portion to the left eye and to the right eye of the user respectively, the stereoscopic VR viewing device enabling the user to view at least one VR game with a stereoscopic and shimmering effect in perception induced by binocular rivalry by applying different colors of monocular stimuli to each eye of the user;
whereby the VR gaming application and the stereoscopic VR viewing device allows the user to play the set of VR games with stereoscopic and shimmering effect.

2. The system of claim 1, wherein the VR game comprises a grid sword game.

3. The system of claim 1, wherein the VR game comprises a clock sword game.

4. The system of claim 1, wherein the VR system comprises a VR headset with an integrated display screen adapted to present separate images to each eye.

5. The system of claim 1, wherein the at least one VR game is a module within a VR gaming application, the VR gaming application configured to execute the at least one VR game and track the user's progress and responses within the central database.

6. The system of claim 5, wherein the at least one VR game comprises a coordination game that requires the user to align objects within a virtual space, the coordination game configured to enhance the user's hand-eye coordination and spatial awareness within the VR environment.

7. The system of claim 5, wherein the at least one VR game comprises a reaction time challenge that presents at least a first visual stimulus in the user's peripheral vision, in addition to puzzles that promote depth perception by requiring the arrangement of objects at varying virtual distances.

8. The system of claim 7, wherein the reaction time challenge VR game assesses and training the user's peripheral vision response and depth discrimination abilities.

9. The system of claim 1, wherein the display screen comprises at least two separate display screens, and/or wherein the display screen comprises an LCD screen, and/or wherein the display screen comprises an LED screen.

10. A stereoscopic vision testing system, comprising:
a display mechanism capable of presenting a first visual stimulus to a user's left eye, and at least a second visual stimulus to the viewer's right eye;
the first visual stimulus and at least second visual stimulus providing the user the ability to detect distinct images in a virtual reality (VR) environment;
wherein the system leverages the user's ability to merge the first visual stimulus and the at least second visual stimulus into at one unified picture; and
wherein the VR environment comprises a virtual reality (VR) unit operably connected to the communication device, the VR unit including an application server residing on a central computer having a processor on which is installed with a VR vision testing application and coupled with a memory unit integrated with a central database, the VR vision testing application comprising a set of VR vision testing applications.

11. The system of claim 10, wherein detection by the user is determined by the user's response in a psychophysical paradigm.

12. The system of claim 11, wherein eye movements are not measured.

13. The system of claim 12, wherein the VR system includes a VR headset with an integrated display screen for presenting the separate images to each eye.

14. The system of claim 12, wherein the first visual stimulus and at least second visual stimulus presented comprise static images or dynamic images.

15. The system of claim 12, wherein the first visual stimulus and at least second visual stimulus comprise dynamic images, said dynamic images presenting a user movement within the VR environment.

16. The system of claim 13, wherein the first visual stimulus and at least second visual stimulus comprise two rivalrous images.

17. The system of claim 14, further comprising an input device adapted to record the user's responses.

18. The system of claim 12, wherein the psychophysical paradigm comprises a choice reaction time test.

19. A method for generating a shimmering effect for multimedia applications, comprising the steps of:
presenting a first visual stimulus of to a user's left eye;
simultaneously presenting at least a second visual stimulus to the viewer's right eye; and
wherein the first visual stimulus and the at least second visual stimulus are configured such that the user's brain cannot resolve the first visual stimulus and second visual stimulus into a single coherent image, thereby producing binocular rivalry and the perception of shimmer in the user.

20. The method of claim 19, wherein the first visual stimulus comprises a first color and the at least second visual stimulus comprises at least a second color, wherein the perception of shimmer is utilized in video or film special effects to enhance visual content.

21. The method of claim 20, wherein the first color comprises a primary color and/or wherein the first visual stimulus and the at least second visual stimulus comprise complementary colors to enhance the perception of shimmer.

22. The method of claim 19, wherein the first visual stimulus and the at least second visual stimulus include geometric shapes and/or wherein the first visual stimulus and the at least second visual stimulus comprise a narrative in video or film.

23. The method of claim 19, further comprising a control mechanism to adjust the intensity of stimulus presented.
